(19) United States
(12) Patent Application Publication
(10) Pub. No.: US 2020/0384178 A1
(43) Pub. Date: Dec. 10, 2020

(54) FACIL CATHETER AND GAUZE BELT

(71) Applicant: Marco Antonio Lucero, Glenarden, MD (US)

(72) Inventor: Marco Antonio Lucero, Glenarden, MD (US)

(21) Appl. No.: 16/501,494

(22) Filed: Nov. 13, 2018

(51) Int. Cl. A61M 1/28

(52) U.S. Cl. CPC A61M 1/285; A61M 2210/10; A61M 2205/02; A61M 2209/088

(57) ABSTRACT

The present invention provides a catheter and gauze band that includes a catheter and gauze retaining sections along the outer and inner surface of the band with a specially adapted snap in device along the band to receive and retain a catheter tube. This present invention also includes methods of use.

All publications including patent documents referred to in this application are incorporated in their entirety for all purposes to the same extent as if each individual publication were individually incorporated by reference.

All headings are for the convenience of the reader and should not be used to limit the meaning of the text following the heading unless so specified.

### TECHNICAL FIELD

The present invention relates to medical devices and more particularly relates to catheter, catheter tubes, gauze, retention and concealment when present on the body.

### BACKGROUND OF THE INVENTION

Peritoneal Dialysis is a method by which soluble waste substances are removed from the body by a process across the peritoneum.

My name is Marco Antonio Lucero, I am the inventor of the Facil Catheter and Gauze Belt, and I am also a peritoneal Dialysis patient.

This process of dialysis involves the introduction of a solution into my peritoneum, the introduced solution then collects soluble waste and thereafter it is drained through a transfer process by a surgically implanted device that originates from the peritoneum on the inside of the body through a tiny incision to the outside by a combination of tubes, catheter and a dialysis device.

The incision point is protected by taping sterile gauze to the body over the incision area. The gauze so taped to the body is required to be changed frequently so as to mitigate bacteria and germ build up and possible contamination and infection. Due to the frequency of gauze replacement, the action of taping irritates and pinches the skin.

As a Dialysis patient, I know the challenges we face. They range from skin irritation, scratches to limited mobility due to the presence of catheter and accessories. I needed more mobility and comfort, so I started searching for a product that will help me eliminate or reduce these problems. The entire product I found, had one general feature, they made provision for retaining the catheter in envelope style pouches. None had a unique way of retaining the catheter to keep it from sliding out of the pouch with increased mobility of the patient or retaining the gauze on the belt so as to eliminate the need for adhesive tapes being constantly plastered on the skin.

U.S. Pat No. 5,853,396 discloses a tuck away belt including a flexible elongated pocket having two panels and a flexible elastic band narrower than the pocket having a hook and loop type attachment at one end and a D ring at the opposite end through which the elastic band is pulled through to attach itself.

I needed a belt that will restrain a tucked away catheter, eliminate the use of adhesive tapes that irritate the skin, pinches and scratches the hair around it and at the same time easy to dismantle for replacement and washing, to give patients like me more comfort and control, so I made the Facil Catheter and Gauze Belt.

### BRIEF SUMMARY OF THE INVENTION

The purpose of this invention is to eliminate irritation, scratching and discomfort of the skin that can occur with the use of adhesive tapes to secure sterile gauze over skin incisions to the body by making a provision a method that safely retains the gauze on a band that can be wrapped around the incision point. This invention also makes provision for easy tuck away, restraining and dismantling of catheter and accessories.

Specifically, the present invention provides a portion along the inner surface of a continuous band that may be worn around the waist, upper chest or in various ways that is adapted to retain sterile gauze on that inner surface by elastic or inelastic means and worn over incision points. The present invention also provides a portion on the same band along the surface that easily retains a catheter tube by a “snap in” process, and a portion along the outer surface of the continuous band a catheter retaining pouch with a “strap in” device that that retains and conceals a catheter by elastic or inelastic means.

### BRIEF DESCRIPTION OF THE DRAWING

FIG. 1: Depicts the front view of the Facil Catheter and Gauze Belt 1, a continuous singular band, worn around the waist 15, may be worn around the chest 16, having a catheter retaining pouch 2 positioned along the outer surface as shown. The catheter retaining pouch 2 has an opening 5 through which the catheter tube 3 lowers into the catheter retaining pouch 2; the catheter tube 3 originates from and is steadied by a snap in device 4, a device that has two upright parallel bars 4A and 4B that extend to form a closed circular loop 4C.

FIG. 2: Depicts the back view of the Facil Catheter and Gauze Belt 1, a continuous singular band when worn around the waist 15, may also be worn around the chest 16 with a fastening system concealed by top stiches 10.

FIG. 3: Depicts a section of the outer surface of the Facil Catheter and Gauze Belt 1, a continuous singular band with a catheter retaining pouch 2 positioned along the outer surface, a snap in device 4 which is able to receives and retain the catheter tube 3 that lowers into the catheter retaining pouch 2 through a top opening 5. The snap in device 4 has two upright parallel bars 4A and 4B which expands when a little downward pressure is applied while placing the catheter tube 3 on top in-between the opening so that the catheter tube 3 is pushed down and retained in the loop 4C

FIG. 4: Depicts a section of the inner surface of the Facil Catheter and Gauze Belt 1, a continuous singular band with a snap in device 4, a top hold down device 6A and a bottom hold down device 6B, a portion 8 in between the hold down mechanism. The hold down devices 6A and 6B are held on to the surface of the Facil Catheter and Gauze Belt 1 by way of side stitches 9.

FIG. 5: Depicts a section of the outer surface of the Facil Catheter and Gauze Belt 1, a continuous singular band with a catheter retaining pouch 2 peeled out to reveal a strap down device 6 and a strapped in catheter 7 lowered into the strap in device 6 by a catheter tube 3 which originates from a snap in device 4 also located on the surface of the Facil Catheter and Gauze Belt 1. The strap in device 6 is secured on the surface of the Facil Catheter and Gauze Belt 1 by two side stitches 9. The catheter tube 3 is snapped into 4 by applying a little downward pressure on top of 4A and 4B, which expands so that the catheter tube 3 is pushed in and retained in the loop 4C

FIG. 6: Depicts a strap 6 that when attached to the surface of the present invention by side stitches 9, leaving a space in the middle, is able to hold down objects, by elastic means. The strap may be elastic or inelastic, held on to the surface of the band by stitching on either side creating a loop that serves as a restraint.

FIG. 7: Depicts the snap in device 4. A rigid hollow device which is slender has two upright parallel sides 4A and 4B that form a loop 4C at the base.

FIG. 8: Depicts the installation of a sterile gauze 13 along the inner surface of the Facil Catheter and Gauze Belt 1, the gauze is inserted into the top hold down device 6A and the bottom hold down device 6B leaving an open space 14 on the gauze that will lap over the body when the Facil Catheter and Gauze Belt 1 is in use.

FIG. 9: Depicts the fastening mechanism for the Facil Catheter and Gauze Belt 1, it includes a series of hooks 11 at one end that may be inserted into a flat D clip 12 to secure the Facil Catheter and Gauze belt when in use. It may be offered in single line configuration of hook 11 and flat D clip 12 or multiple line configuration of hook 11, 17 and 18.

### DETAILED DESCRIPTION OF THE DRAWING

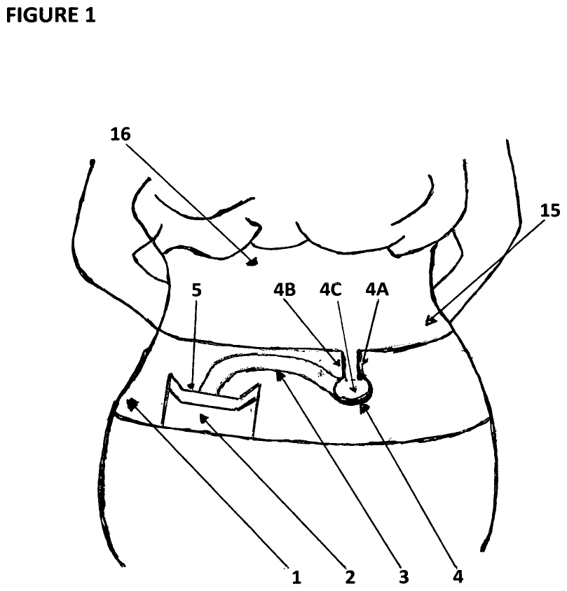

Referring to FIG. 1, the disclosed Facil Catheter and Gauze Belt 1 includes an elastic or inelastic continuous band, may be worn around the waist 15 or chest 16 or other convenient ways with a catheter retaining pouch 2. The catheter retaining pouch 2 located on the outer surface of the Facil Catheter and Gauze Belt 1, is able to conceal a catheter lowered into it by way of a catheter tube 3 which originates from, and is held steady by a snap in device 4. It is evident that the present invention is easy to use because the catheter tube 3 is able to be received and retained by a snap in device 4, a method achieved by applying a downward pressure on the upright bars 4A and 4B of the snap in device 4 causing them to expand and receive the Catheter tube 3 which is pushed into position 4C which is a circular loop formed at the base by the upright bar 4A and upright bar 4B. This device dearly eliminates the need for the Catheter and accessories to be dismantled every time the belt is to be changed or washed. The catheter tube originating and retained by the snap in device 4 lowers into the catheter retaining pouch 2 by way of a top opening 5.

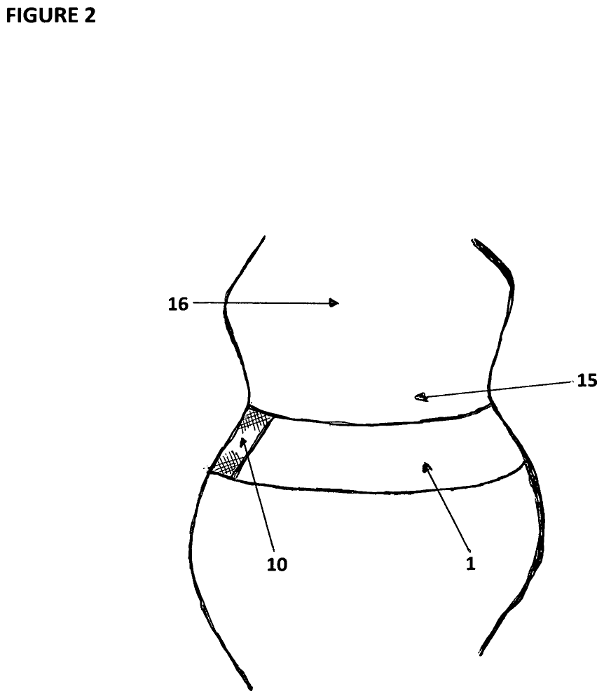

Referring to FIG. 2, the disclosed Facil Catheter and Gauze Belt 1, worn around the waist 15 may be worn around the chest 16 or other convenient ways includes an elastic or inelastic continuous singular band secured together by a fastening mechanism concealed by top stitches 10. The position of the fastening device may vary when the Facil Catheter and Gauze Belt 1 is worn, the fastening mechanism may be offered in variety of ways, and may be positioned on either side, the back or a combination of ways. The band is machine washable, it may be offered in various sizes to accommodate a variety of waist lines for adults and children or may be offered in a one size fits all for adults and children. When offered in a one size fits all, may be equipped with adjustable securing device to accommodate variety of waist lines. When offered in sizes, they may generally correspond to extra small, small, medium, large, extra large and extra extra large. The present invention encompasses adults and children sizes, children being classified as newborn to the age of twelve and adult as thirteen and above.

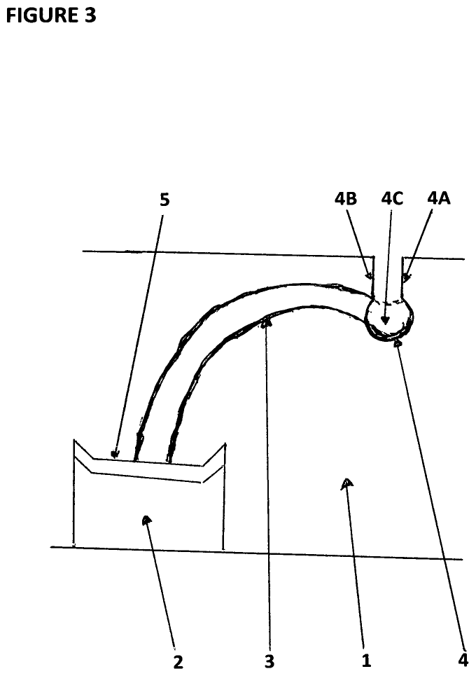

Referring to FIG. 3, the disclosed Facil Catheter and Gauze Belt 1 includes a portion along the outer surface with a catheter retaining pouch 2 which may be made of elastic or inelastic material, of a dimension big enough to receive and retain a catheter. The catheter retaining pouch 2 functions to receive and conceal a catheter lowered into the catheter retaining pouch 2 by way of a catheter tube 3. This function does not however limit its use to catheter alone but other devices within the medical field that may be so retained and concealed. The catheter tube 3 originates from and is retained by a snap in device 4. The snap in device 4 receives the catheter tube 3 when the catheter tube 3 is placed over the top of the snap in device 4 and by applying a downward pressure on the upright bar 4A and 4B while placing the catheter tube in the space between the two bars. The pressure causes the bars to expand, creating a path for the catheter tube to be pushed into the circular loop 4C formed at the base by the upright bars where it is retained. The Catheter tube 3 may be dismantling by applying an upward pressure on the catheter tube 3 while it is being pulled up, which will also cause the upright bar 4A and upright bar 4B to expand creating a path for the catheter tube to be disengaged from the device. The catheter retaining pouch 2 has a top opening 5 through which the catheter tube 3 lowers into the catheter retaining pouch 2. The catheter retaining pouch may be constructed entirely of an elastic material or may be constructed in part by a combination of an elastic or inelastic material or entirely by an inelastic material. It may be offered in various sizes.

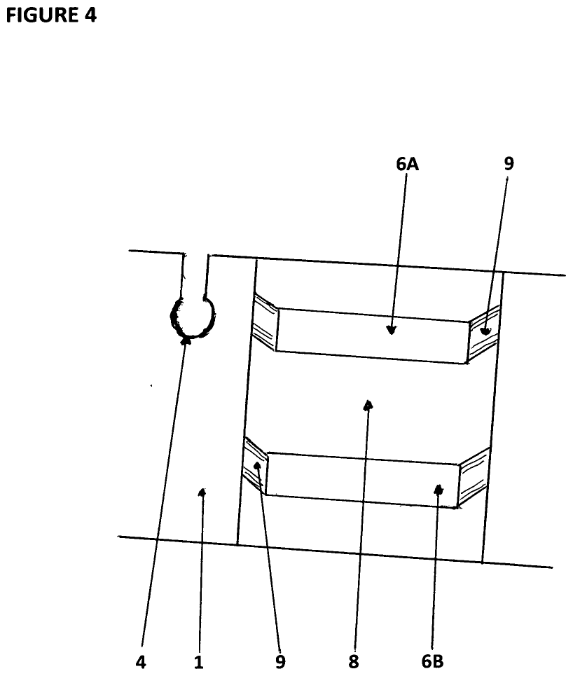

Referring to FIG. 4, the disclosed Facil Catheter and Gauze Belt 1 includes a portion along the inner surface a section for retaining sterile gauze. The portion has a section equipped with a top hold down device 6A and a bottom hold down device 6B, they may be elastic or inelastic, held to the band by stitching 9 at both ends thus creating a hollow in the middle that is able to receive and retain by insertion a gauze. In between the top hold down device 6A and the bottom hold down device 6B also includes a portion 8 which is void unless a gauze is installed. To the left of the section adapted for gauze retention on the inner surface is the snap in device 4, adapted to receive the tube. The snap in device 4 may be offered in various forms and or sizes.

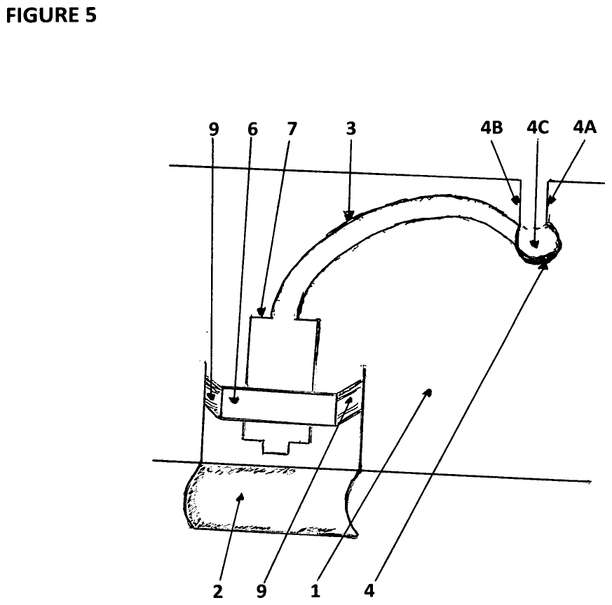

Referring to FIG. 5, the disclosed Facil Catheter and Gauze Belt 1 includes an elastic or inelastic continuous singular band, a snap in device 4 which may be offered in various sizes and forms and is able to retain the catheter tube 3 by placing the catheter tube 3 in on top of the device, in-between the two upright bars 4A and 4B while applying a downward pressure which will cause the bars to dilate, expand and create a larger pathway for the catheter tube 3 to be pushed down to the loop 4C at the base. The catheter tube 3 may be removed by applying an upward pressure on the catheter tube 3 while pulling it up, which will cause the upright bars 4A and 4B to dilate and create a larger path for the catheter tube 3 to be pulled up and out of the device. The catheter tube 3 is able to lower the catheter 7 into the catheter retaining pouch 2 which is peeled out to reveal the catheter strap in device 6, attached to the band by two side stitches 9 leaving a space along the middle for a catheter 7 to be inserted and so retained.

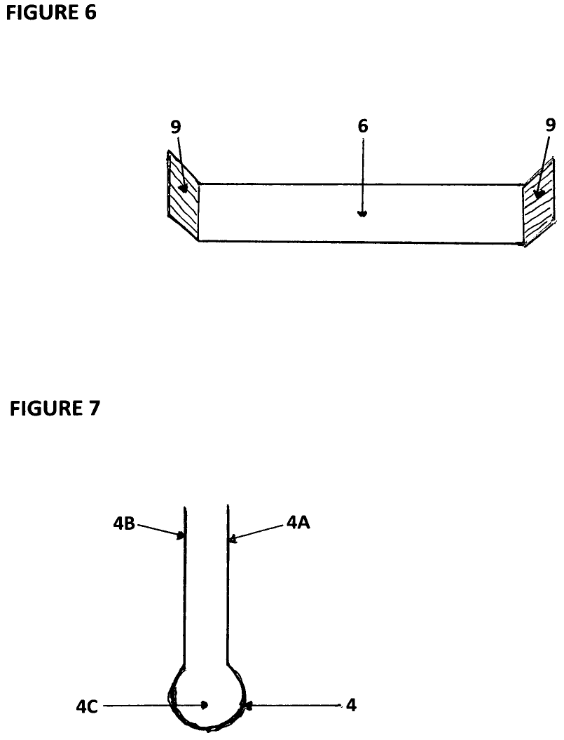

Referring to FIG. 6, the hold down device for the disclosed invention may be made of elastic or inelastic material. It may be offered in various forms and sizes where present on this particular invention, specifically as 6, 6A or 6B on the outer surface of the band and or the inner surface of the band. The strap is held down on the band by two side stiches, one stitch 9 on the left side and another stitch 9 on the right side. The hold down device may be offered in various forms to include various adhesive materials or restraining methods.

Referring to FIG. 7 the snap in device 4 may be a rigid or flexible material, may be made out of plastic or cloth may also be made in part or wholly of some or all of the material with two upright parallel bars 4A and 4B stretching from top with a space in between to a form a circular loop 4C at the base, open at the tip but dosed at the base. The upright bars 4A and 4B are able to expand or dilate when a little downward or upward pressure is applied to the space in between and also contract when no pressure is applied. The device may be offered in various sizes and shapes.

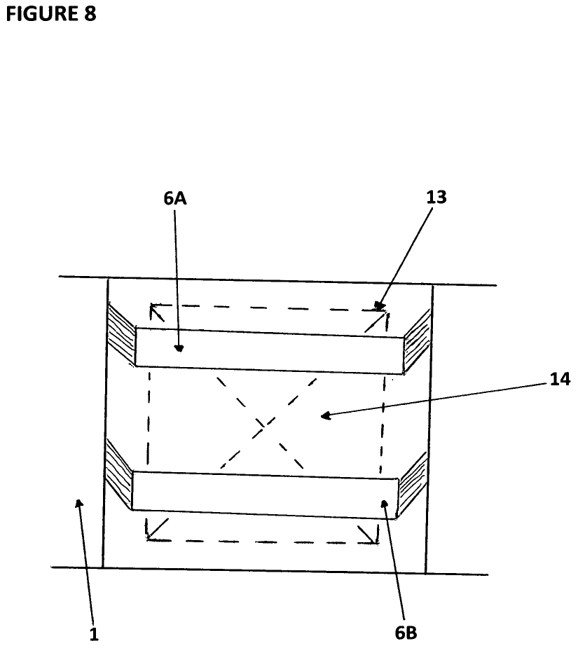

Referring to FIG. 8, the disclosed Facil Catheter and Gauze Belt 1 includes a portion on the inner surface adapted to receive and retain gauze 13. The gauze 13 is installed by pushing or inserting the top edge into the top hold down device 6A and pushing or inserting the bottom edge into the bottom hold down device 6B. The hold down mechanism may be made of elastic or inelastic material or may be made from other forms of adhesive material. The gauze 13 while inserted, has a space 14 around the middle section in between the top hold down device 6A and the bottom hold down device 6B. The gauze 13 so inserted, presses across the body when worn or when this present invention is in use and works to protect the incision point thereby eliminating the use of adhesive tapes. Thus this present invention eliminates the use of adhesive tapes on the body that can cause scratching, pinching and irritates the skin.

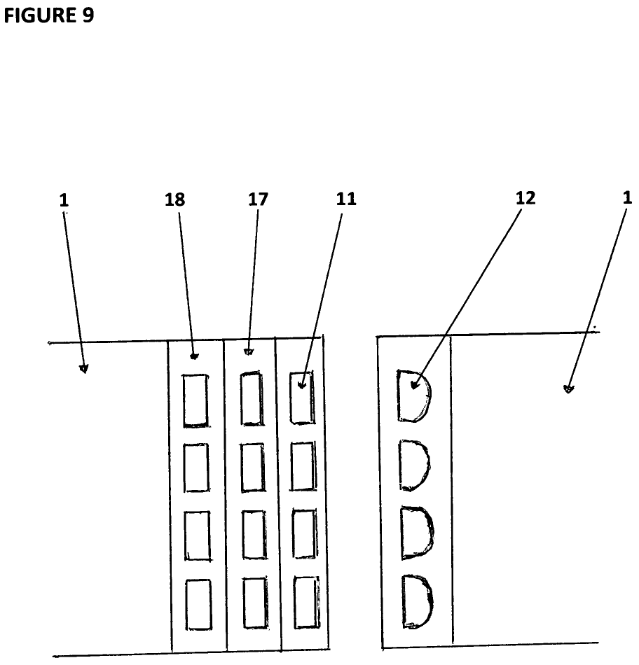

Referring to FIG. 9, the disclosed Facil Catheter and Gauze Belt 1, a continuous singular band may be elastic or inelastic is secured and held together by a fastening device when in use. The fastening device consists of a series of hooks 11 in a single line configuration at one end and a series of a flat D ring 12 in a single line configuration at the other end. The hook 11 at the one end, is clipped to the flat D ring 12 at the other end to create a locking mechanism. The hook 11 may be offered in a single line configuration or in a multiple line 17 and 18 configuration. The hook 11 may be offered in various forms and may be made of various materials. The flat ring may be offered in various forms, sizes and may be made of various materials. The fastening device may be positioned in various ways along the band. It may be positioned as a unit on either side, the back or a combination of units on either side and or back.

1. A catheter and gauze belt comprising of;
a) a continuous singular band, elastic or inelastic with a gauze retaining portion along the inner surface of the said band
b) a continuous singular band elastic or inelastic with a catheter restraining device inside a catheter retaining pouch and
c) a continuous singular band, elastic or inelastic with a snap in device to retain a catheter tube
Whereas the said gauze retaining device, catheter retaining pouch and the restraining device inside the pouch and the snap in device are constructed in whole, part or a combination thereof from adhesive like materials, Velcro, elastic, inelastic, nylon, cotton, plastic or pseudo plastic materials.
